SEQUENCE LISTING (6 sequences)

SEQ ID NO: 1 - DNA, 20 bp, Artificial Sequence
  Sparwasser, et al. (1998) Eur. J. Immunol. 28 2045-2054.
tccatgacgttcctgatgct

SEQ ID NO: 2 - DNA, 24 bp, Artificial Sequence
  Hartmann and Krieg (2000) J. Immunol. 164944- 953.
tcgtcgttttgtcgttttgtcgtt

SEQ ID NO: 3 - DNA, 23 bp, Artificial Sequence
  Hartmann and Krieg (2000) J. Immunol. 164944- 953.
tcgtcgttttgtcgttttgtcgt

SEQ ID NO: 4 - DNA, 30 bp, Artificial Sequence
  Yamamoto, et al. (1994) Jpn. J. Cancer Res. 85775-779.
accgataccggtgccggtgacggcaccacg

SEQ ID NO: 5 - DNA, 30 bp, Artificial Sequence
  Yamamoto, et al. (1994) Jpn. J. Cancer Res. 85 775-779.
accgataacgttgccggtgacggcaccacg

SEQ ID NO: 6 - DNA, 30 bp, Artificial Sequence
  Yamamoto, et al. (1994) Jpn. J. Cancer Res. 85 775-779.
accgatgacgtcgccggtgacggcaccacg

(19) United States
(12) Patent Application Publication
(10) Pub. No.: US 2003/0003579 A1
(43) Pub. Date: Jan. 2, 2003

(54) Dendritic cells; methods

(76) Inventors: Norimitsu Kadowaki, Kyoto (JP); Yong-Jun Liu, Palo Alto, CA (US)

Correspondence Address: DNAX RESEARCH, INC., LEGAL DEPARTMENT, 901 CALIFORNIA AVENUE, PALO ALTO, CA, 94304, US

(21) Appl. No.: 10/011,635

(22) Filed: Oct. 22, 2001

Related U.S. Application Data
(60) Provisional application No. 60/243,232, filed on Oct. 24, 2000.

(51) Int. Cl. C12N 15/87; C12N 5/08

(52) U.S. Cl. 435/455; 435/372

(57) ABSTRACT

Methods of effecting particular dendritic cell subsets. In particular, methods for inducing responses in defined subsets of dendritic cells are provided.

This application claims benefit of U.S. Provisional Patent Application No. 60/243,232, filed Oct. 24, 2000.

### FIELD OF THE INVENTION

The invention relates generally to methods of modulating physiology of certain defined subsets of dendritic cells, more particularly, to methods of regulating production of various interferons by subsets of dendritic cells.

### BACKGROUND

The circulating component of the mammalian circulatory system comprises various cell types, including red and white blood cells of the erythroid and myeloid cell lineages. See, e.g., Rapaport (1987) Introduction to Hematology (2d ed.) Lippincott, Philadelphia, Pa.; Jandl (1987) Blood: Textbook of Hematology, Little, Brown and Co., Boston, Mass.; and Paul (ed. 1993) Fundamental Immunology (3d ed.) Raven Press, N.Y.

Dendritic cells (DCs) are the most potent of antigen presenting cells. See, e.g., Paul (ed. 1998) Fundamental Immunology 4th ed., Raven Press, NY. Antigen presentation refers to the cellular events in which a proteinaceous antigen is taken up, processed by antigen presenting cells (APC), and then recognized to initiate an immune response. The most active antigen presenting cells have been characterized as the macrophages (which are direct developmental products from monocytes), dendritic cells, and certain B cells. DCs are highly responsive to inflammatory stimuli such as bacterial lipopolysaccharides (LPS) and cytokines such as tumor necrosis factor alpha (TNFα). The presence of cytokines and LPS can induce a series of phenotypic and functional changes in DC that are collectively referred to as maturation. See, e.g., Banchereau and Schmitt (eds. 1995) Dendritic Cells in Fundamental and Clinical Immunology Plenum Press, NY.

Dendritic cells can be classified into various categories, including: interstitial dendritic cells of the heart, kidney, gut, and lung; Langerhans cells in the skin and mucous membranes; interdigitating dendritic cells in the thymic medulla and secondary lymphoid tissue; and blood and lymph dendritic cells. Although dendritic cells in each of these compartments are CD45+ leukocytes that apparently arise from bone marrow, they may exhibit differences that relate to maturation state and microenvironment. Maturational changes in DCs include, e.g., silencing of antigen uptake by endocytosis, upregulation of surface molecules related to T cell activation, and active production of a number of cytokines including TNFα and IL-12. Upon local accumulation of TNFα, DCs migrate to the T cell areas of secondary lymphoid organs to activate antigen specific T cells.

Many factors have been identified which influence the differentiation process of precursor cells, or regulate the physiology or migration properties of specific cell types. See, e.g., Mire-Sluis and Thorpe (1998) Cytokines Academic Press, San Diego; Thomson (ed. 1998) The Cytokine Handbook (3d ed.) Academic Press, San Diego; Metcalf and Nicola (1995) The Hematopoietic Colony Stimulating Factors Cambridge University Press; and Aggarwal and Gutterman (1991) Human Cytokines Blackwell. These factors provide yet unrecognized biological activities, e.g., on different untested cell types.

However, dendritic cells are poorly characterized, both in terms of responses to soluble factors, and many of their functions and mechanisms of action. The absence of knowledge about the physiological properties and responses of these cells limits their understanding. Thus, medical conditions where regulation, development, or physiology of dendritic cells is unusual remain unmanageable. The present invention addresses these issues.

### SUMMARY OF THE INVENTION

The present invention is based, in part, upon the surprising discovery of specificity of nucleic acid effects on different DC subsets. In particular, Applicants have determined that two separate classes of nucleic acid adjuvants, the CpGs and poly I:Cs, target distinct types of cells. This insight thus directs one to distinct targets for identifying receptors for the respective adjuvants. The functional receptor for the CpGs would be on IPCs, e.g., pDC2 cells; while the receptor for the poly I:Cs would be on the myeloid lineage dendritic cells.

The present invention provides, in one embodiment, methods comprising contacting an IPC with an effective amount of CpG nucleic acid, thereby inducing: maturation of the IPC to a dendritic cell; and IFNα production by the IPC upon viral stimulation. Certain forms include those wherein the IFNα production is at least 1000 pg/ml/10⁵ cells; the DC is a potent antigen presenting cell; or the CpG is ODN 1668; ODN 2117; ODN 2006; ODN ACC-30; ODN AAC-30; or ODN GAC-30.

A second series of methods are provided comprising contacting a myeloid lineage dendritic cell with an effective amount of poly I:C nucleic acid, thereby inducing: IFNα production by the DC; IL-12 production by the DC; and/or maturation of the DC. Preferably, the DC matures to a potent antigen presenting cell; the: IFNα production is at least 25 pg/ml/10⁵ cells; or IL-12 production is at least 25 pg/ml/10⁵ cells.

Also provided are methods to detect a receptor for a nucleic acid comprising: screening for the presence of a receptor for a CpG on an IPC cell; or screening for the presence of a poly I:C on a myeloid lineage DC. For example, in the method screening for a receptor for a CpG on an IPC cell, typically, there is screening for: blocking of CpG mediated effect with a monoclonal antibody against an antigen found on IPC cells, thereby identifying an antigen through which CpG signaling is mediated; or for direct binding of the CpG to receptors expressed by IPC cells. Appropriate CpG mediators are ODN 1668; ODN 2117; ODN 2006; ODN ACC-30; ODN AAC-30; or ODN GAC-30; and a particularly likely receptor is a TLR, including TLR10 or TLR6. Screening can take advantage of the CpG mediated effects of IFNα production or pDC2 maturation.

Alternatively, methods are provided screening for a receptor for poly I:C on a myeloid lineage DC, comprising screening for: blocking of poly I:C mediated effect with a monoclonal antibody against an antigen found on myeloid lineage DC, thereby identifying an antigen through which poly I:C signaling is mediated; or direct binding of the poly I:C to receptors expressed by myeloid lineage DC. Particularly useful will be where the poly I:C mediated effect is IFNα production, IL-12 production, or DC maturation.

### DETAILED DESCRIPTION OF THE INVENTION

### Outline

I. General

A. DC, Type I IFNs, and IPC

B. nucleic acid effectors

II. Adjuvant effects

A. pDC2; maturation to APC; CpG effects

B. myeloid DC; cytokines and APC; poly I:C effects

C. combination

Ill. Uses

IV. Receptor Identification and Isolation

I. General

Dendritic cells (DCs) represent heterogeneous populations of hematopoietic-derived cells that display potent ability to induce primary T cell activation, polarization, and in certain circumstances tolerance. See Sousa, et al. (1999) Curr. Op. Immunol. 11:392-399; Sallusto and Lanzavecchia (1999) J. Exp. Med. 189:611-614; Banchereau and Steinman (1998) Nature 392:245-252; Cella, et al. (1997) Curr. Opin. Immunol. 9:10-16; and Steinman (1991) Annu. Rev. Immunol. 9:271-296. The distinct capacity of DCs to induce immunity versus tolerance or Th1 versus Th2 responses depends on their maturation stage (Cella, et al. (1997) Curr. Opin. Immunol. 9:10-16; and Kalinski, et al. (1999) Immunol. Today 20:561-567), signals that induce or inhibit DC maturation (Cella, et al. (1997) Curr. Opin. Immunol. 9:10-16; and Kalinski, et al. (1999) Immunol. Today 20:561-567; d'Ostiani, et al. (2000) J. Exp. Med. 191:1661-1674), as well as the lineage origin of DCs (Pulendran, et al. (1999) Proc. Nat'l Acad. Sci. USA 96:1036-1041; Reis e'Sousa, et al. (1999) Curr. Opin. Immunol. 11:392-399; Maldonado-Lopez, et al. (1999) J. Exp. Med. 189:587-592; Arpinati, et al. (2000) Blood 95:2484-2490; Liu and Blom (2000) Blood 95:2482-2483; and Shortman (2000) Immunol. Cell Biol. 78:161-165). Dendritic cells, which are the primary antigen presenting cells (APC), are thought to be very important in the recognition of pathogens through pattern recognition receptors, leading to a primary immune response. Their roles in innate and active immune responses are still incompletely understood.

A lymphoid DC developmental pathway was suggested by the finding that mouse thymic lymphoid precursors can give rise to both T cells and CD8⁺CD11b⁻ DCs. Ardavin, et al. (1993) Nature 362:761-763; and Shortman, et al. (1998) Immunol. Rev 165:39-46. In addition, a well-established myeloid DC pathway giving rise to CD8⁻CD11b⁺ DCs has been defined. Inaba, et al. (1992) J. Exp. Med. 176:1693-1702; Inaba, et al. (1993) Proc. Nat'l Acad. Sci. USA 90:3038-2042; and Young and Steinman (1996) Stem Cells 14:376-287. Recent studies suggest that CD8^{+CD}11b⁻ lymphoid DCs and CD8⁻CD 11b⁺ myeloid DCs may have different functions in T cell activation/tolerance or Th1/Th2 differentiation. Pulendran, et al. (1999) Proc. Nat'l Acad. Sci. USA 96:1036-1041; Maldonado-Lopez, et al. (1999) J. Exp. Med. 189:587-592; Suss and Shortman (1996) J. Exp. Med. 183:1789-1796; Kronin, et al. (1997) Int. Immunol. 9:1061-1064; Stumbles, et al. (1998) J. Exp. Med. 188:2019-2031; Ohteki, et al. (1999) J. Exp. Med. 189:1981-1986; Thomson, et al. (1999) J. Leukoc. Biol. 66:322-330; Iwasaki and Kelsall (1999) J. Exp. Med. 190:229-239; and Khanna, et al. (2000) J. Immunol. 164:1346-1354.

In humans, two distinct populations of dendritic cell precursors have been identified in the blood. Monocytes (pre-DC1), which belong to the myeloid lineage, differentiate into immature DC1 after 5 days of culture in granulocyte colony-stimulating factor (GM-CSF) and IL-4. Sallusto and Lanzavecchia (1994) J. Exp. Med. 179:1109-1118; and Romani, et al. (1994) J. Exp. Med. 180:83-93. Upon CD40-Ligand activation, immature myeloid DC1 undergo maturation and produce large amounts of IL-12. Cella, et al. (1996) J. Exp. Med. 184:747-752; and Koch, et al. (1996) J. Exp. Med. 184:741-746. The mature DC1 induced by CD40-Ligand are able to polarize naive CD4⁺ T cells into Th1 cells. Rissoan, et al. (1999) Science 283:1183-1186. The second type of DC precursor cells, pre-DC2 (previously known as plasmacytoid T/monocytes) are characterized by a surface phenotype (CD4⁺IL-3Rα⁺⁺CD45RA⁺HLA-DR⁺ lineage markers⁻ and CD11c⁻), and at the ultrastructural level resemble immunoglobulin-secreting plasma cells. Grouard, et al. (1997) J. Exp. Med. 185:1101-1111; and Facchetti, et al. (1999) Histopathology 35:88-89. Several lines of evidence suggest that pre-DC2s are of lymphoid origin: i) pre-DC2 lack expression of the myeloid antigens CD11c, CD13, CD33, and mannose receptor (Grouard, et al. (1997) J. Exp. Med. 185:1101-1111; and Res, et al. (1999) Blood 94:2647-2657), ii) pre-DC2 isolated from the thymus, express the lymphoid markers CD2, CD5, and CD7 (Res, et al. (1999) Blood 94:2647-2657), iii) pre-DC2 have little phagocytic activity (Grouard, et al. (1997) J. Exp. Med. 185:1101-1111), iv) pre-DC2 do not differentiate into macrophages following culture with GM-CSF and macrophage-colony stimulating factor (M-CSF) (Grouard, et al. (1997) J. Exp. Med. 185:1101-1111), v) pre-DC2 express pre-TCR alpha transcripts (Res, et al. (1999) Blood 94:2647-2657; and Bruno, et al. (1997) J. Exp. Med. 185:875-884), and vi) development of pre-DC2, T, and B cells, but not myeloid DC, are blocked by ectopic expression of inhibitor of DNA binding (Id)2 or Id3. Pre-DC2 differentiate into immature DC2 when cultured with monocyte conditional medium (O'Doherty, et al. (1994) Immunology 82:487-493), IL-3 (Rissoan, et al. (1999) Science 283:1183-1186; Grouard, et al. (1997) J. Exp. Med. 185:1101-1111; and Olweus, et al. (1997) Proc. Nat'l Acad. Sci. USA 94:12551-12556), IFN-α/β and tumor necrosis factor (TNF)-α or viruses, like Herpes Simplex Virus or Influenza virus (Kadowaki, et al. (2000) J. Exp. Med. 192:219-226). Upon CD4⁺-Ligand activation, immature DC2 undergo maturation (Grouard, et al. (1997) J. Exp. Med. 185:1101-1111), but produce only low levels of IL-12 (Rissoan, et al. (1999) Science 283:1183-1186). Mature DC2 are able to polarize naive CD4⁺ T cells into a Th2 phenotype (Arpinati, et al. (2000) Blood 95:2484-2490; and Rissoan, et al. (1999) Science 283:1183-1186). Recent studies showed that the pre-DC2 are the elusive natural interferon producing cells (IPC), capable of producing high amounts of IFN-α/β upon viral stimulation (Siegal, et al. (1999) Science 284:1835-1837; and Cella, et al. (1999) Nature Med. 5:919-923). Taken together, pre-DC2/IPCs represent a unique hematopoietic lineage, capable of performing crucial functions both in innate and in adapted immunity.

Two classes of nucleic acids, bacterial DNA containing unmethylated CpG motifs (see, e.g., Krieg U.S. Pat. No. 6,008,200) and double-stranded RNA (dsRNA) in viruses, induce the production of type I interferon (IFN), which contributes to immunostimulatory effects of these microbial molecules. Certain sequences within bacterial (or invertebrate) DNA (CpG motifs) have been shown to exhibit immunomodulatory effects. These motifs are unmethylated CpG dinucleotides within particular sequence contexts, often in 12-20mer forms, with the motif: 5′-pu-pu-CG-py-py-3′. See, e.g., Hartmann and Krieg (2000) J. Immunol. 164:944-952. The CpGs may be recognized by the vertebrate immune system as foreign bacterial or viral DNA because they are unmethylated. They can induce a spectrum of innate, humoral, and cellular immune responses, e.g., activation of NK cells, stimulation of B cell proliferation, costimulation of T cells, and effects on DC, including upregulation of MHC-II, CD40, CD86, and induction of TNFα, IL-6, and IL-12 secretion. CpGs exhibit an adjuvant effect comparable to complete Freund's adjuvant, but also seem to induce innate protective responses. Thus, the CpGs favor a Th1 type response in a mouse, e.g., protection in a tumor vaccination model, CTL mediated antiviral protection, and reorientation from a Th2 to a Th1 response in an asthma model. Synthetic oligodeoxynucleotides (ODN) typically mimic the effects of the microbial DNA.

It is important to determine which cells produce type I IFN in response to CpG DNA and dsRNA. CD4⁺IL-3Rα^{high}CD3-CD11c⁻ type 2 dendritic cell precursors (pre-DC2) were identified as main producers of type I IFN in human blood in response to viral infections, e.g, Herpes Simplex Virus or Influenza virus. Here is addressed whether pre-DC2 are also the target of CpG DNA and dsRNA for type I IFN production. Oligodeoxynucleotides containing particular palindromic CpG motifs induced pre-DC2, but not CD11c⁺ blood immature DC or monocytes, to produce IFN-α. In contrast, a synthetic dsRNA, polyinosinic polycytidylic acid (poly I:C), induced CD11c⁺ DC, but not pre-DC2 or monocytes, to produce IFN-α/β. These data indicate that poly I:C and CpG DNA stimulate different types of cells to produce type I IFN and that it is important to select oligodeoxynucleotides containing particular CpG motifs in order to induce pre-DC2 to produce type I IFN, which may play a key role in strong adjuvant effects of CpG DNA.

Natural Interferon-α producing cells (IPC) are key effector cells in anti-viral innate immunity. These cells produce up to 1000 times more IFN-α than other blood cell types in response to viral stimulation. IPCs also have the capacity to become dendritic cells, which are key antigen presenting cells (APC) in the induction of T cell mediate immune responses.

Upon viral stimulation, the natural IFN-α/β producing cells (IPCs, also known as pre-DC2) in human blood and peripheral lymphoid tissues rapidly produce very large amounts of IFN-α/β. After performing this innate anti-viral immune response, IPCs can differentiate into dendritic cells and strongly stimulate T cell mediated adaptive immune responses.

The innate immune system has the capacity to recognize invariant molecular patterns shared by microbial pathogens. See, e.g., Medzhitov and Janeway (1997) Curr. Opin. Immunol. 9:4-9. Recent studies have revealed that this recognition is a crucial step to induce effective immune responses. A major mechanism by which microbial components augment immune responses is to stimulate antigen-presenting cells (APC), especially dendritic cells (DC), to produce proinflammatory cytokines and to express high levels of costimulatory molecules for T cells. See also Reis e Sousa, et al. (1999) Curr. Opin. Immunol. 11:392-399. These activated DC subsequently initiate primary T cell responses and dictate the types of T cell-mediated effector functions. See Banchereau and Steinman (1998) Nature 392:245-252.

II. Adjuvant effects

Two classes of nucleic acids, namely (i) bacterial CpG DNA that contains immunostimulatory unmethylated CpG dinucleotides within specific flanking bases (referred to as CpG motifs; see Krieg (2000) Curr. Opin. Immunol. 12:35-43; and Krieg US Pat. 6,008,200) and (ii) double-stranded RNA (dsRNA) synthesized by various types of viruses (Jacobs and Langland (1996) Virology 219:339-349), represent important members of the microbial components that enhance immune responses.

Recent studies have shown that oligodeoxynucleotides (ODNs) containing CpG motifs (see Sparwasser, et al. (1998) Eur. J. Immunol. 28:2045-2054; and Hartmann, et al. (1999) Proc. Nat'l Acad. Sci. USA 96:9305-9310) and synthetic dsRNA, i.e., polyinosinic polycytidylic acid (poly I:C; see Celia, et al. (1999) J. Exp'l Med. 189:821-829; and Verdijk, et al. (1999) J. Immunol. 163:57-61), are capable of inducing DC to produce proinflammatory cytokines and to express high levels of costimulatory molecules.

A series of studies have shown that bacterial DNA or synthetic ODNs containing unique palindromic CpG motifs induce human PBMC (Yamamoto, et al. (1994) Jpn J. Cancer Res. 85:775-779) and mouse spleen cells (Yamamoto, et al. (1992) J. Immunol. 148:4072-4076) to produce type I interferon (IFN-α/β). See Yamamoto, et al. (2000) Curr. Top. Microbiol. Immunol. 247:23-39.

Poly I:C was originally synthesized as a potent inducer of type I IFN. See De Clercq (1981) Methods Enzymol. 78:227-236; and Levy (1981) Methods Enzymol. 78:242-251. The typical structure is similar to one polyl strand hybridized to a polyC strand on a ribonucleic acid backbone. Modified molecular structures should target the same cellular receptors. These homologs probably mimic the structure of double stranded viral RNAs which induce production of the type I interferons.

Type I IFN plays an essential role in antiviral innate immunity and is widely used to treat viral hepatitis and various types of cancers. Pfeffer, et al. (1998) Cancer Res. 58:2489-2499. These effects appear to be due to direct inhibition of viral replication in infected cells and to pleiotropic immunomodulating activity of type I IFN, such as (i) enhancing cytotoxicity of NK cells and macrophages (Pfeffer, et al. (1998) Cancer Res. 58:2489-2499), (ii) inducing T cell activation (Sun, et al. (1998) J. Exp'l Med. 188:2335-2342), (iii) maintaining survival of activated T cells (Marrack, et al. (1999) J. Exp'l Med. 189:521-530), (iv) stimulating human CD4⁺ T cells to produce a Th1 cytokine IFN-γ (Demeure, et al. (1994) J. Immunol. 152:4775-4782), and (v) inducing the expression of TNF-related apoptosis-inducing ligand (TRAIL) on T cells and thereby enhancing T cell cytotoxicity (Kayagaki, et al. (1999) J. Exp'l Med. 189:1451-1460). Thus, CpG DNA and poly I:C are believed to be promising adjuvants for vaccination against infections and cancers owing to their DC-stimulating and type I IFN-inducing capacity.

To understand the mechanisms underlying the induction of type I IFN by CpG DNA and poly I:C and to increase their efficacy as immunological adjuvants, it is important to determine the target cells of CpG DNA and poly I:C for type I IFN induction. Two groups have recently shown that the main producers of type I IFN in human blood, designated as natural type I IFN-producing cells (IPC), are identical to CD4⁺IL-3Rα^{high}CD3-CD11c⁻ DC2 precursors (pre-DC2; see Siegal, et al. (1999) Science 284:1835-1837; and Celia, et al. (1999) Nature Med. 5:919-923), which differentiate into DC in response to IL-3 (Grouard, et al. (1997) J. Exp'l Med. 185:1101 -1111) or viruses (Kadowaki, et al. (2000) J. Exp'l Med. 192:219-226). Pre-DC2/IPC produce from about 200 to 1,000 (e.g., 400, 600, 800) times more type I IFN than do CD 11c⁺ blood immature DC, monocytes, and monocyte-derived DC in response to viral stimulation. Siegal, et al. (1999) Science 284:1835-1837. Herein is addressed whether pre-DC2 are the blood cells that produce type I IFN in response to CpG DNA and poly I:C. It is shown that (i) pre-DC2, but not CD11c⁺ DC or monocytes, produce type I IFN in response to CpG ODNs containing particular palindromic sequences and that (ii) CD11c⁺ DC, but not pre-DC2 or monocytes, produce type I IFN in response to poly I:C.

One of the main effects of CpG DNA (Yamamoto, et al. (2000) Curr. Top. Microbiol. Immunol. 247:23-39) and poly I:C (De Clercq (1981) Methods Enzymol. 78:227-236; and Levy (1981) Methods Enzymol. 78:242-251) is the induction of type I IFNs. In addition to the essential role of type I IFNs in antiviral innate immunity (Pfeffer, et al. (1998) Cancer Res. 58:2489-2499), they appear to be key cytokines to induce effective adaptive immunity due to their pleiotropic effects on various types of immune cells. Pfeffer, et al. (1998) Cancer Res. 58:2489-2499; Sun, et al. (1998) J. Exp'l Med. 188:2335-2342; Marrack, et al. (1999) J. Exp'l Med. 189:521-530; Demeure, et al. (1994) J. Immunol. 152:4775-4782; and Kayagaki, et al. (1999) J. Exp'l Med. 189:1451-1460. Therefore, it is important to determine which cells produce type I IFN in response to CpG DNA or poly I:C in order to understand the mechanisms by which these nucleic acids augment immune responses and to exploit their ability as immunological adjuvants. This study was directed to the question of whether pre-DC2/IPC, the most potent producers of type I IFN in response to viruses (Siegal, et al. (1999) Science 284:1835-1837; and Celia, et al. (1999) Nature Med. 5:919-923), are the target of CpG DNA and poly I:C for type I IFN production. It was found that (i) CpG ODNs containing certain palindromic sequences induce pre-DC2, but not CD11c⁺ DC, to produce type I IFN and that (ii) poly I:C stimulates CD11c⁺ DC, but not pre-DC2, to produce type I IFN.

Hartmann, et al., screened en extensive series of CpG ODNs to find the ones with highest immunostimulatory activity for human cells. Hartmann and Krieg (2000) J. Immunol. 164:944-953; and Hartmann, et al. (2000) J. Immunol. 164:1617-1624. They and others found that CpG ODN 2006 most potently activates human B cells (Hartmann and Krieg (2000) J. Immunol. 164:944-953), monocytes (Bauer, et al. (1999) Immunology 97:699-705), and DC (Hartmann, et al. (1999) Proc. Nat'l Acad. Sci. USA 96:9305-9310). In line with these findings, 2006 induced marked upregulation of CD80 and CD86 on pre-DC2. However, this CpG ODN did not induce pre-DC2 to produce detectable levels of IFN-α. In contrast, another class of CpG ODNs AAC-30 and GAC-30, which have been shown to induce human PBMC (Yamamoto, et al. (1994) Jpn J. Cancer Res. 85:775-779) and mouse spleen cells (Yamamoto, et al. (1992) J. Immunol. 148:4072-4076) to produce type I IFN, induced pre-DC2 to produce IFN-α. On the other hand, monocytes or CD11c⁺ DC did not produce detectable levels of IFN-α in response to 2006, AAC-30, or GAC-30. These data suggest that (i) pre-DC2 are the cell type that produces IFN-α in response to CpG DNAs and that (ii) it is important to select CpG DNAs containing particular sequences in order to induce pre-DC2 to produce IFN-α. In addition, the finding that 2006 induced pre-DC2 to upregulate CD80 and CD86 but not to produce IFN-α suggest that CpG DNA may induce pre-DC2 to differentiate into DC and to produce IFN-α through distinct signaling pathways.

The present studies suggest that CD11c⁺ DC are the main blood cell type that produce a significant amount of type I IFN in response to poly I:C. Related nucleic acid polymers have also been reported to have antiviral or immune effects. See, e.g., Stebbing and Eaton U.S. Pat. No. 4,152,350; Hutchison and Eaton U.S. Pat. No. 3,935,185; Kraska U.S. Pat. No. 4,193,999; Yano, et al. U.S. Pat. No. 5,298,614; Einck U.S. Pat. No. 5,763,417; Field, et al. U.S. Pat. No. 4,388,306; Arimura, et al. U.S. Pat. No. 4,313,938; and Lampson, et al. U.S. Pat. No. 4,124,702. CD11c⁺ DC and fibroblasts produced similar levels of IFN-β. Since the number of fibroblasts in tissues is probably larger than that of CD11c⁺ DC, the main producers of type I IFN in response to poly I:C and dsRNA may be fibroblasts, but not blood cells, as has been reported. De Clercq (1981) Methods Enzymol. 78:227-236; and Levy (1981) Methods Enzymol. 78:242-251. It has recently been shown that poly I:C induces maturation of monocyte-derived immature DC. Celia, et al. (1999) J. Exp'l Med. 189:821-829; Verdijk, et al. (1999) J. Immunol. 163:57-61. Whereas CD11c⁺ DC appear to be myeloid-derived DC because they express myeloid markers (O'Doherty, et al. (1994) Immunology 82:487-493), pre-DC2-derived DC may be lymphoid-derived DC because they lack myeloid markers (O'Doherty, et al. (1994) Immunology 82:487-493) and express mRNA of pre-T receptor α chain (Res, et al. (1999) Blood 94:2647-2657). Thus, poly I:C may stimulate myeloid-derived but not lymphoid-derived DC.

Combinations of the CpGs and poly I:Cs should affect both populations of cells, leading to induction of the effector functions along both pathways. The effects would be expected, at least, to be additive, and may even be synergistic.

III. Uses

IPC will be important in a number of therapeutic and research applications. See, e.g., Kadowaki, et al. (2000) J. Expt'l Med. 192:219-226; and Liu and Blom (2000) Blood 95:2482-2483. They will be used in cellular therapy for viral infections and diseases, e.g., HIV or hepatitis, and for tumor therapies.

CpG DNA has pleiotropic effects on the immune system through activating APC, i.e., B cells, macrophages, and D C. Krieg (2000) Curr. Opin. Immunol. 12:35-43; and Krieg U.S. Pat. No. 6,008,200. In particular, a strong Th1-inducing effect of CpG DNA makes it a useful immunological adjuvant to treat infectious diseases (Klinman, et al. (1999) Immunity 11:123-129), cancers (Weiner (2000) Curr. Top. Microbiol. Immunol. 247:157-170), and allergic diseases (Kline (2000) Curr. Top. Microbiol. Immunol. 247:211-225). It is likely that type I IFN induced by CpG DNA contributes to the immunostimulatory effects of CpG DNA through various mechanisms, e.g., enhancing NK cell activity (Yamamoto, et al. (1992) J. Immunol. 148:4072-4076), inducing T cell activation (Sun, et al. (1998) J. Exp'l Med. 188:2335-2342), and enhancing IFN-γ production by T cells (Demeure, et al. (1994) J. Immunol. 152:4775-4782). Therefore, CpG ODNs that induce pre-DC2 to produce type I IFN may be suitable reagents for clinical application. Deoxyribonuclease-resistant phosphorothioate forms of AAC-30 and GAC-30 do not have a type I IFN-inducing effect on pre-DC2. Designing phosphorothioate forms of CpG ODNs having such an effect may be an important future direction of CpG immunology.

The IPC will produce natural interferons, and can substitute for administration of the interferons in treatment of medical conditions. Methods are available to isolate large quantities of pDC2 cells, which will allow for further analysis. See, e.g., U.S. Ser. No. 60/234,142. this allows a source material from which to identify and clone the receptor for CpG, providing reagents for further study and understanding of the pathway by which the CpGs effect their adjuvant activities. This will ultimately provide means to regulate IFN-α production.

CpGs may also have anticancer effects, which includes protection against infectious diseases after irradiation or chemotherapy. Effects on NK cells, as tumor adjuvants, or DC based vaccination strategies may also exist.

The present invention provides teachings which allow modulation of physiology mediated by defined dendritic cell subsets. Populations of substantially homogeneous IPCs will have important utility in research, diagnostic, or therapeutic environments.

Effects on various cell types may be indirect, as well as direct. A statistically significant change in the effects on cells will typically be at least about 10%, preferably 20%, 30%, 50%, 70%, 90%, or more. Effects of greater than 100%, e.g., 130%, 150%, 2×, 3×, 5×, etc., will often be desired.

The present invention will be useful in the treatment of medical conditions or diseases associated with innate or viral immunity. See, e.g., Frank, et al. (eds. 1995) Samter's Immunologic Diseases. 5th Ed., vols. I-II, Little, Brown and Co., Boston, Mass.

The adjuvant substances described may be combined with other treatments of the medical conditions described herein, e.g., an antibiotic, antifungal, antiviral, immune suppressive therapeutic, immune adjuvant, analgesic, anti-inflammatory drug, growth factor, cytokine, vasodilator, or vasoconstrictor. See, e.g., the Physician's Desk Reference, both prescription and non-prescription compendiums. Preferred combination therapies include the materials or reagents with various anti-tumor or anti-infective agents.

Standard immunological techniques are described, e.g., in Hertzenberg, et al. (eds. 1996) Weir's Handbook of Experimental Immunology vols. 1-4, Blackwell Science; Coligan (1991) Current Protocols in Immunology Wiley/Greene, NY; and Methods in Enzymology volumes 70, 73, 74, 84, 92, 93, 108, 116, 121, 132, 150, 162, and 163.

To prepare pharmaceutical or sterile compositions including, e.g., these nucleic acid analogs, the material is admixed with a pharmaceutically acceptable carrier or excipient which is preferably inert. Preparation of such pharmaceutical compositions is known in the art, see, e.g., Remington's Pharmaceutical Sciences and U.S. Pharmacopeia: National Formulary, Mack Publishing Company, Easton, Pa. (1984). Typically, therapeutic compositions are sterile.

The nucleic acid homologs may be administered orally or parenterally, preferably intravenously. Since such homologs might be immunogenic they are preferably administered slowly, either by a conventional IV administration set or from a subcutaneous depot, e.g. as taught by Tomasi, et al., U.S. Pat. No. 4,732,863.

When administered parenterally the therapeutics will typically be formulated in a unit dosage injectable form (solution, suspension, emulsion) in association with a pharmaceutically acceptable parenteral vehicle. Such vehicles are inherently nontoxic and nontherapeutic. The antagonist may be administered in aqueous vehicles such as water, saline, or buffered vehicles with or without various additives and/or diluting agents. Alternatively, a suspension, such as a zinc suspension, can be prepared to include the peptide. Such a suspension can be useful for subcutaneous (SQ), intradermal (ID), or intramuscular (IM) injection. The proportion of therapeutic entity and additive can be varied over a broad range so long as both are present in effective combination amounts. The therapeutic is preferably formulated in purified form substantially free of aggregates, proteins, endotoxins, and the like, at appropriate concentrations, e.g., about 5 to 30 mg/ml, preferably 10 to 20 mg/ml. Preferably, the endotoxin levels are less than 2.5 EU/ml. See, e.g., Avis, et al. (eds. 1993) Pharmaceutical Dosage Forms: Parenteral Medications 2d ed., Dekker, NY; Lieberman, et al. (eds. 1990) Pharmaceutical Dosage Forms: Tablets 2d ed., Dekker, NY; Lieberman, et al. (eds. 1990) Pharmaceutical Dosage Forms: Disperse Systems Dekker, NY; Fodor, et al. (1991) Science 251:767-773; Coligan (ed.) Current Protocols in Immunology; Hood, et al. Immunology Benjamin/Cummings; Paul (ed. 1997) Fundamental Immunology 4th ed., Academic Press; Parce, et al. (1989) Science 246:243-247; Owicki, et al. (1990) Proc. Nat'l Acad. Sci. USA 87:4007-4011; and Blundell and Johnson (1976) Protein Crystallography, Academic Press, New York.

Selecting an administration regimen for a therapeutic agonist or antagonist depends on several factors, including the serum or tissue turnover rate of the therapeutic, the immunogenicity of the therapeutic, or the accessibility of the target cells. Preferably, an administration regimen maximizes the amount of therapeutic delivered to the patient consistent with an acceptable level of side effects. Accordingly, the amount of therapeutic delivered depends in part on the particular agonist or antagonist and the severity of the condition being treated.

Determination of the appropriate dose is made by the clinician, e.g., using parameters or factors known in the art to affect treatment or predicted to affect treatment. Generally, the dose begins with an amount somewhat less than the optimum dose and it is increased by small increments thereafter until the desired or optimum effect is achieved relative to any negative side effects.

The phrase “effective amount” means an amount sufficient to effect a desired response, or to ameliorate a symptom or sign of the target condition.

Typical mammalian hosts will include mice, rats, cats, dogs, and primates, including humans. An effective amount for a particular patient may vary depending on factors such as the condition being treated, the overall health of the patient, the method, route, and dose of administration and the severity of side affects. Preferably, the effect will result in a change in quantitation of at least about 10%, preferably at least 20%, 30%, 50%, 70%, or even 90% or more. When in combination, an effective amount is in ratio to a combination of components and the effect is not necessarily limited to individual components alone.

An effective amount of therapeutic will modulate the symptoms typically by at least about 10%; usually by at least about 20%; preferably at least about 30%; or more preferably at least about 50%. Such will result in, e.g., statistically significant and quantifiable changes in the numbers of cells being affected. This may be an increase or decrease in the numbers of target cells appearing within a time period or target area.

The present invention provides reagents which will find use in therapeutic applications as described elsewhere herein. See, e.g., Berkow (ed.) The Merck Manual of Diagnosis and Therapy, Merck & Co., Rahway, N.J.; Thorn, et al. Harrison's Principles of Internal Medicine, McGraw-Hill, NY; Gilman, et al. (eds. 1990) Goodman and Gilman's: The Pharmacological Bases of Therapeutics, 8th Ed., Pergamon Press; Remington's Pharmaceutical Sciences, 17th ed. (1990), Mack Publishing Co., Easton, Penn; Langer (1990) Science 249:1527-1533; and Merck Index, Merck & Co., Rahway, N.J.

Antibodies to marker proteins may be used for the identification or sorting of cell populations expressing those markers. Methods to sort such populations are well known in the art, see, e.g., Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

IV. Receptor Identification and Isolation

With biological activities defined for the two forms of nucleic acid effectors, screening methods for identifying the receptors for the effectors become available. Identification of the receptor is important because it allows screening for small molecule agonists or antagonists of the nucleic acid effectors. A number of different strategies are readily apparent.

Neutralizing antibodies may be generated which block the effector biological activities. Antibodies are raised against cell surface antigens from the target cells, e.g., pDC2, IPC, or myeloid lineage DC. The antibodies, preferably monoclonal antibodies, are tested for ability to block the effector mediated activity. Screens for maturation or IFNα production are readily developed. Antibodies which bind to the receptor are likely to block the interaction of the nucleic acid homolog with the receptor. Thus, the antibody is likely to block the effector function. The receptor may then be expression cloned using the antibody.

Likely receptors include the TLRs, especially TLR10 and TLR6. See, e.g., U.S. Ser. No. 60/207,558. Specifically, these genes can be tested directly or indirectly for interaction with the nucleic acid homologs. Cell based assays may be developed.

Direct binding assays may also be used with, e.g., labeled CpGs or poly I:Cs. In vitro assays may be developed, though cell based assays may be devised.

The broad scope of this invention is best understood with reference to the following examples, which are not intended to limit the inventions to the specific embodiments.

### EXAMPLES

### I. General Methods

Some of the standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Greene/Wiley, New York; Innis, et al. (eds.)(1990) PCR Protocols: A Guide to Methods and Applications Academic Press, N.Y. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Methods in Enzymology, vol. 182, and other volumes in this series; manufacturer's literature on use of protein purification products, e.g., Pharmacia, Piscataway, N.J., or Bio-Rad, Richmond, Calif.; and Coligan, et al. (eds.) (1995 and periodic supplements) Current Protocols in Protein Science, John Wiley & Sons, New York, N.Y. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering Principle and Methods 12:87-98, Plenum Press, N.Y.; and Crowe, et al. (1992) QlAexpress: The High Level Expression & Protein Purification System QIAGEN, Inc., Chatsworth, Calif.

Standard immunological techniques are described, e.g., in Hertzenberg, et al. (eds. 1996) Weir's Handbook of Experimental Immunology vols. 1-4, Blackwell Science; Coligan (1991) Current Protocols in Immunology Wiley/Greene, NY; and Methods in Enzymology volumes. 70, 73, 74, 84, 92, 93, 108, 116, 121, 132, 150, 162, and 163.

FACS analyses are described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

II. Production of OligoDeoxyNucleotides (ODNs)

The following ODNs were purchased from Research Genetics:

1668, TCCATGACGTTCCTGATGCT (Sparwasser, et al. (1998) Eur. J. Immunol. 28:2045-2054);

2117, T*CGT*CGTTTTGT*CGTTTTGT*CGTT (*C, methylated cytidine) (Hartmann and Krieg (2000) J. Immunol. 164:944-953);

2006, TCGTCGTTTTGTCGTTTTGTCGT (Hartmann and Krieg (2000) J. Immunol. 164:944-953);

ACC-30, ACCGATACCGGTGCCGGTGACGGCACCACG (Yamamoto, et al. (1994) Jpn J. Cancer Res. 85:775-779);

AAC-30, ACCGATAACGTTGCCGGTGACGGCACCACG (Yamamoto, et al. (1994) Jpn J. Cancer Res. 85:775-779);

GAC-30, ACCGATGACGTCGCCGGTGACGGCACCACG (Yamamoto, et al. (1994) Jpn J. Cancer Res. 85:775-779).

Underlines indicate palindromic sequences. ODNs 1668, 2117, and 2006 are phosphorothioate forms, and ACC-30, AAC-30, and GAC-30 are phosphodiester forms. The phosphorothioate ODNs were added at 0 h at 0.1 μM or 1 μM, and the phosphodiester ODNs were added at 0, 4, and 16 h, 5 μM at each time point, to compensate for their degradation by deoxyribonuclease activity in media.

III. Isolation and culture of cells

Monocytes, CD11c⁺ DC, and pre-DC2 were isolated from human peripheral blood. See Grouard, et al. (1997) J. Exp'l Med. 185:1101-1111; and Kadowaki, et al. (2000) J. Exp'l Med. 192:219-226. The cells were cultured for 24 h in RPMI1640 containing 10% FCS at 2×10⁴/200 μl in round-bottom 96-well culture plates in the presence of ODNs or 50 μg/ml poly I:C (Sigma). Adult dermal fibroblasts (Clonetics) were maintained as recommended by the company, and were stimulated with 50 μg/ml poly I:C for 24 h at 7.5×10⁴/ml in 12-well culture plates.

IV. Analysis of viability of pre-DC2 stimulated with ODNs or poly I:C Pre-DC2 cultured for 24 h without stimulation or with ODNs or poly I:C were stained with propidium iodide, and were analyzed with a FACScan® flow cytometer (Becton Dickinson). After cell debris was excluded by an appropriate forward scatter threshold, the percentages of propidium iodide-negative cells were calculated.

V. Flow cytometric analysis of the expression of CD80 and CD86

Freshly isolated pre-DC2 and pre-DC2 stimulated with ODNs for 24 h were stained with PE-conjugated anti-CD80 (L307.4, Becton Dickinson), PE-conjugated anti-CD86 (2331, Pharmingen), or an isotype control antibody. CD11c⁺ DC cultured for 24 h without stimulation or with poly I:C were stained with FITC-conjugated anti-CD80 (L307.4), FITC-conjugated anti-CD86 (2331), or an isotype control antibody. The cells were analyzed with a FACScan® flow cytometer. Dead cells were excluded by staining with propidium iodide.

VI. Quantitation of cytokines by ELISA

An IFN-α ELISA kit, an IL-12 ELISA kit (Biosource International), and an IFN-β ELISA kit (FUJIREBIO) were used to analyze cytokine production.

VII. CpG ODNs but not poly I:C maintain the survival of pre-DC2 and induce them to differentiate into DC

Pre-DC2 were cultured for 24 h without stimulation, with the indicated ODNs, or with 50 μg/ml poly I:C. ACC-30, AAC-30, and GAC-30 were added at 5 μM. After cell debris was excluded by an appropriate forward scatter threshold, the percentages of propidium iodide-negative cells were measured by flow cytometry

Without any stimuli, the majority of pre-DC2 rapidly die within 24 h. Grouard, et al. (1997) J. Exp'l Med. 185:1101-1111; and Kadowaki, et al. (2000) J. Exp'l Med. 192:219-226. It was first examined whether different CpG ODNs and poly I:C prevent pre-DC2 from dying. Three types of CpG ODNs were used: (i) 2006, which upregulates the expression of costimulatory molecules on human blood DC (Hartmann, et al. (1999) Proc. Nat'l Acad. Sci. USA 96:9305-9310) and B cells (Hartmann and Krieg (2000) J. Immunol. 164:944-953), (ii) AAC-30 and GAC-30, which have palindromic CpG motifs and stimulate human PBMC (Yamamoto, et al. (1994) Jpn J. Cancer Res. 85:775-779) and mouse spleen cells (Yamamoto, et al. (1992) J. Immunol. 148:4072-4076) to produce type I IFN, and (iii) 1668, which stimulates mouse DC to express high levels of costimulatory molecules and to produce proinflammatory cytokines TNF-α, IL-6, and IL-12 (Sparwasser, et al. (1998) Eur. J. Immunol. 28:2045-2054).

1 μM CpG ODN 2006 efficiently blocked the death of pre-DC2. The same concentration of control CpG ODN 2117 having the same sequence except for methylated cytidines blocked the death of pre-DC2 to a lesser extent than 2006. In the presence of a lower concentration (0.1 μM) of 2117, most pre-DC2 died, whereas a significant number of pre-DC2 survived in the presence of 0.1 μM 2006. These results are consistent with the finding that methylation of cytosine residues significantly diminishes immunostimulatory activity of CpG DNA. Hartmann, et al. (1999) Proc. Nat'l Acad. Sci. USA 96:9305-9310. Another group of CpG ODNs AAC-30 and GAC-30 also maintained the survival of pre-DC2, while ACC-30, which has been shown to lack the ability to induce human PBMC to produce type I IFN (Yamamoto, et al. (1994) Jpn J. Cancer Res. 85:775-779), was less efficient. Although 1 μM CpG ODN 1668 has been shown to activate mouse DC (Sparwasser, et al. (1998) Eur. J. Immunol. 28:2045-2054), the same concentration of 1668 did not maintain the survival of pre-DC2. This is consistent with the finding that CpG DNAs with strong stimulatory activity in the mouse system do not necessarily activate human immune cells. Hartmann and Krieg (2000) J. Immunol. 164:944-953; and Bauer, et al. (1999) Immunology 97:699-705. In contrast to the CpG ODNs immunostimulatory for human cells, poly I:C did not maintain the survival of pre-DC2.

Freshly isolated pre-DC2 and pre-DC2 cultured with the CpG ODNs for 24 h were stained with PE-conjugated anti-CD80 or anti-CD86 mAbs. The upregulation of CD80 and CD86 expression on pre-DC2 is a hallmark of their differentiation into DC. Grouard, et al. (1997) J. Exp'l Med. 185:1101-1111; and Kadowaki, et al. (2000) J. Exp'l Med. 192:219-226. One μM unmethylated CpG ODN 2006 and methylated CpG ODN 2117 strongly upregulated the expression of CD80 and CD86. AAC-30 and GAC-30 also upregulated CD80 and CD86, albeit to a lesser extent than 2006 and 2117.

Taken together, these data indicate that CpG ODNs containing appropriate sequences efficiently maintain the survival of pre-DC2 and induce them to differentiate into DC. In marked contrast, poly I:C does not stimulate pre-DC2 to survive and to differentiate into DC.

VIII. Distinct CpG ODNs but not poly I:C induce pre-DC2 to produce IFN-α

It was examined whether CpG ODNs and poly I:C induce pre-DC2 to produce IFN-α. Cells were stimulated with various CpG ODNs (2117, 2006, 1668: 1 μM, ACC-30, AAC-30, GAC-30: 5 μM) for 24 h, and the concentrations of IFN-α in the supernatants were measured by ELISA. Typically, cytokine is accumulated from cultures of about 2×10⁴ cells in 200 μl cultured for 24 h. Although 2006 strikingly upregulated CD80 and CD86, this CpG ODN as well as 2117 and 1668 did not induce pre-DC2 to produce detectable levels of IFN-α. In contrast, AAC-30 and GAC-30 induced pre-DC2 to produce large amounts of IFN-α (AAC-30 508-1806 pg/ml, GAC-30 931-1362 pg/ml). ACC-30 induced pre-DC2 to produce much smaller amounts of IFN-α (18-161 pg/ml). None of the CpG ODNs used here induced CD11c⁺ DC and monocytes to produce detectable levels of IFN-α. In line with the finding that poly I:C did not maintain the survival of pre-DC2, this reagent did not induce pre-DC2 to produce a detectable level of IFN-α. These data indicate that ODNs containing particular CpG motifs, but not poly I:C, induce pre-DC2 to produce IFN-α.

IX. Poly I:C stimulates CD11c⁺ myeloid DC to produce IFN-α/β and IL-12 and to undergo maturation

CD11c⁺ DC cultured for 24 h without stimulation or with 50 μg/ml poly I:C were stained with FITC-conjugated anti-CD80 or anti-CD86 mAbs. Although poly I:C did not induce pre-DC2 to survive or to produce IFN-α, this reagent has been shown to induce human monocyte-derived DC to mature and to produce IL-12. Cella, et al. (1999) J. Exp'l Med. 189:821-829; and Verdijk, et al. (1999) J. Immunol. 163:57-61. Thus, it was asked whether poly I:C stimulates CD11c⁺ myeloid DC (O'Doherty, et al. (1994) Immunology 82:487-493) to produce type I IFN and IL-12 and to undergo maturation. Poly I:C induced CD11c⁺ DC, but not pre-DC2 or monocytes, to produce low but significant levels of IFN-α (e.g., 24.1-97.9 pg/ml), IFN-β (e.g., 33.6-166.5 pg/ml), and IL-12 (e.g., 17.7-137.7 pg/ml). Poly I:C stimulated fibroblasts produced similar levels of IFN-β (e.g., 28.6-48.5 pg/ml). Poly I:C strongly upregulated the expression of CD80 and CD86 on CD11c⁺ DC during 24 h culture. Thus, poly I:C stimulated CD11c⁺ myeloid DC, but not pre-DC2, to produce type I IFN and IL-12 and to become mature DC.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method comprising contacting an IPC with an effective amount of CpG nucleic acid, thereby inducing:
a) maturation of said IPC to a dendritic cell; and
b) IFNα production by said IPC upon viral stimulation.

2. The method of claim 1, wherein said IFNα production is at least 1000 pg/ml/10⁵ cells.

3. The method of claim 1, wherein said DC is a potent antigen presenting cell.

4. The method of claim 1, wherein said CpG is ODN 1668; ODN 2117; ODN 2006; ODN ACC-30; ODN MC-30; or ODN GAC-30.

5. The method of claim 4, wherein said CpG is ODN AAC30; ODN 2117; or ODN 2006.

6. A method comprising contacting a myeloid lineage dendritic cell with an effective amount of poly I:C nucleic acid, thereby inducing:
a) IFNα production by said DC;
b) IL-12 production by said DC; and/or
c) maturation of said DC.

7. The method of claim 6 wherein said inducing is: inducing:
a) IFNα production by said DC;
b) IL-12 production by said DC; and
c) maturation of said DC.

8. The method of claim 7, wherein said DC matures to a potent antigen presenting cell.

9. The method of claim 6, wherein said:
a) IFNα production is at least 25 pg/ml/10⁵ cells; or
b) IL-12 production is at least 25 pg/ml/ml/10⁵ cells.

10. The method of claim 7, wherein said:
a) IFNα production is at least 25 pg/ml/10⁵ cells; or
b) IL-12 production is at least 25 pg/ml/10⁵ cells.

11. A method to detect a receptor for a nucleic acid comprising:
a) screening for the presence of a receptor for a CpG on an IPC cell; or
b) screening for the presence of a poly I:C on a myeloid lineage DC.

12. The method of claim 11, screening for a receptor for a CpG on an IPC cell, comprising screening for:
a) blocking of CpG mediated effect with a monoclonal antibody against an antigen found on IPC cells, thereby identifying an antigen through which CpG signaling is mediated; or
b) direct binding of said CpG to receptors expressed by IPC cells.

13. The method of claim 12, wherein said CpG is ODN 1668; ODN 2117; ODN 2006; ODN ACC-30; ODN AAC-30; or ODN GAC-30.

14. The method of claim 12, screening for a receptor for a CpG on an IPC cell, comprising screening for blocking of CpG mediated effect with a monoclonal antibody against an antigen found on IPC cells, thereby identifying an antigen through which CpG signaling is mediated.

15. The method of claim 14, wherein said antigen is a TLR, including TLR10 or TLR6.

16. The method of claim 14, wherein said CpG mediated effect is IFNα production or pDC2 maturation.

17. The method of claim 11, screening for a receptor for poly I:C on a myeloid lineage DC, comprising screening for:
a) blocking of poly I:C mediated effect with a monoclonal antibody against an antigen found on myeloid lineage DC, thereby identifying an antigen through which poly I:C signaling is mediated; or
b) direct binding of said poly I:C to receptors expressed by myeloid lineage DC.

18. The method of claim 17, screening for a receptor for poly I:C on a myeloid lineage DC, comprising screening for blocking of poly I:C mediated effect with a monoclonal antibody against an antigen found on myeloid lineage DC, thereby identifying an antigen through which poly I:C signaling is mediated.

19. The method of claim 18, wherein said poly I:C mediated effect is IFNα production, IL-12 production, or DC maturation.
